(19) United States
(12) Patent Application Publication
(10) Pub. No.: US 2025/0113813 A1
(43) Pub. Date: Apr. 10, 2025

(54) Fluid Reservoir Having Controlled Wick Immersion Level

(71) Applicant: Thermacell Repellents, Inc., Bedford, MA (US)

(72) Inventors: Wender Wang, Bedford, MA (US); Fernando L. Castro, Bedford, MA (US)

(73) Assignee: Thermacell Repellents, Inc., Bedford, MA (US)

(21) Appl. No.: 18/834,787

(22) PCT Filed: Jan. 31, 2023
(86) PCT No.: PCT/US2023/012000

§ 371 Date: Jul. 31, 2024

Related U.S. Application Data
(60) Provisional application No. 63/304,894, filed on Jan. 31, 2022.

(51) Int. Cl. A01M 1/20; A01M 29/12

(52) U.S. Cl. CPC A01M 1/2061; A01M 29/12

(57) ABSTRACT

A vapor emitting device has a main body with a wick. The main body includes a material reservoir and a wick reservoir. The material reservoir is configured to contain a volatilizable material and a vacuum volume. The vacuum volume supports at least a partial vacuum at a predetermined level. The material reservoir has a base surface surrounded by reservoir walls with a top surface including an inlet formed on the top surface. The wick reservoir extends from the inlet through the material reservoir and towards the base surface. The wick reservoir has a first end and a second end. The first end is adjacent to the inlet and includes an inlet orifice. The second end is adjacent to the base surface and has a material orifice. The wick is disposed through the inlet and the wick reservoir with an air gap formed between the wick and the wick reservoir.

### CROSS-REFERENCE TO RELATED APPLICATIONS

This application claims the benefit of U.S. Provisional Application No. 63/304,894, filed Jan. 31, 2022, the disclosure of which is incorporated herein by reference in its entirety.

### BACKGROUND OF THE INVENTION

This invention relates in general to vapor emitting devices generally and, in particular, to a reservoir and wick structure that draws fluid material from the reservoir up through the wick by capillary action uniformly and consistently in response to a changing fluid volume within the reservoir.

Volatilizable fluids dispensed into the environment by wicks are well known in the art. Typically, a wick is placed in a volume of fluid material contained in a reservoir. The material is drawn up through the wick by capillary action and the released into the environment through various means, such as convection, forced convection, evaporation, and/or heat-aided volatilization. The amount of wick contacting the material changes as the fluid is drawn up by the wick. As the amount of wick material immersed in the fluid decreases, the fluid uptake rate is reduced, and the vapor emission decreases. This condition results in decreased performance of the device due the wick releasing an inconsistent amount of material over time. Thus, it would be desirable to provide a reservoir structure that improves vapor delivery from a wick in contact with a volatilizable fluid.

### SUMMARY OF THE INVENTION

This invention relates to devices that emit vapors of a volatilizable fluid contained in a reservoir and transported through a wick to the environment. In certain embodiments, the wick is exposed to heat to generate the vapor emission. In one embodiment, the device is an insect repeller that emits vapors of metofluthrin or similar compounds.

The invention controls the liquid surface level in the space surrounding the wick. In one embodiment, the reservoir comprises two vessels or volumes: a material reservoir holding the bulk of the volatilizable material and a “wick reservoir” into which the wick projects. When the liquid surface level in the wick reservoir drops below a certain threshold, air enters the material reservoir allowing liquid to transfer from the material reservoir to the wick reservoir until the level in the wick reservoir raises to submerge an end of the wick reservoir. Then liquid transfer stops and the surface level in the wick chamber is controlled. In another embodiment, the wick extends into a depression or wick chamber formed in the bottom of the material reservoir. In certain aspects of the invention the wick may be covered in an impervious sheath or tube that restricts fluid exposure to the portion of the wick in the wick chamber. In certain embodiments, providing a close-fitting cylinder-to-wick projection in the bottom of the refill container will assure the wick is immersed in liquid for almost all of the capacity of the refill bottle. Further, if the bottle is shaped like a saucer so small changes in surface level yield large changes in volume, wicking consistency may be enhanced.

In certain embodiments, a vapor emitting device has a main body with a wick. The main body includes a material reservoir and a wick reservoir. The material reservoir is configured to contain a volatilizable material and a vacuum volume. The vacuum volume supports at least a partial vacuum which may be at a predetermined level relative to fluid contained within the material reservoir. The material reservoir has a base surface surrounded by reservoir walls with a top surface. The top surface includes an inlet formed on the top surface. The wick reservoir extends from the inlet through the material reservoir and towards the base surface. The wick reservoir has a first end and a second end. The first end is adjacent to the inlet and includes an inlet orifice. The second end is adjacent to the base surface and has a material orifice. The wick is disposed through the inlet and the wick reservoir with an air gap formed between the wick and the wick reservoir.

In certain embodiments, a vapor emitting device has a main body with a wick. The main body includes a material reservoir and a wick reservoir. The reservoir is configured to contain a volatilizable material and a vacuum volume. The vacuum volume supports at least a partial vacuum at a predetermined level. The reservoir has a base surrounded by reservoir walls with a top surface. The base includes a depression formed on the base. The depression defines a wick immersion volume. The top surface has an inlet formed on the top surface. The wick reservoir extends through the material reservoir and towards the base surface from the inlet. The wick reservoir has a first end and a second end. The first end is adjacent to the inlet and includes an inlet orifice. The second end is adjacent to the wick immersion volume and includes a material orifice. The wick is disposed through the inlet, the wick reservoir, and the wick immersion volume with an air gap formed between the wick and the wick reservoir.

In certain embodiments, a vapor emitting device has a main body. The main body includes a material reservoir. The material reservoir is configured to contain a volatilizable material. The material reservoir has a base surface surrounded by material reservoir walls with a top surface. The base surface includes a depression formed on the base surface. The depression defines a wick immersion volume. The top surface has an inlet formed on the top surface. The wick is disposed through the inlet and the wick immersion volume.

In certain embodiments, a vapor emitting device comprises a main body, a wick reservoir, and a wick. The main body has a material reservoir configured to contain a volatilizable material and a vacuum volume. The vacuum volume supports at least a partial vacuum. The wick reservoir extends through the material reservoir and has an inlet in fluid communication with an ambient atmosphere and a material orifice in fluid communication with the volatilizable material. The wick is disposed through the wick reservoir and into contact with the volatilizable material. The wick and wick reservoir define an air gap configured to permit the ambient atmosphere to accumulate in the vacuum volume such that the volatilizable material is drawn into the wick. In certain aspects, the wick reservoir forms a fluid barrier between a portion of the wick and the material reservoir.

The wick reservoir defines a wick immersion level that controls a length of the wick in contact with the volatilizable material for capillary uptake. The wick immersion level is established between a material orifice of the wick reservoir and a submerged end of the wick. A first pressure plane is defined between an interface of the volatilizable material and the vacuum volume and a second pressure plane is defined in proximity to the material orifice of the wick reservoir. In one aspect of the invention, the length of the wick in contact with the volatilizable material is in a range of about 15% to about 25% of an overall length of the wick.

In certain embodiments, the material reservoir of the vapor emitting device has a base surface includes a wick immersion volume defining the wick immersion level. In certain embodiments, the wick immersion volume is configured as a depression extending from the base surface. In other embodiments, the material reservoir is a first material reservoir and the wick immersion volume is a second material reservoir and a dividing wall separates the first material reservoir from the second material reservoir. In one aspect of the invention, the dividing wall includes at least one fluid port to permit fluid to pass from the first material reservoir to the second material reservoir.

In other embodiments, the wick reservoir is a separate tubular structure extending from the inlet and the air gap is defined by one of a plurality of flutes or a plurality of tubes formed on the wick reservoir.

An insect repeller orients the wick within a heating element to thermally vaporize the volatilizable material in the wick. In certain embodiments, the volatilizable material is metofluthrin.

Various aspects of this invention will become apparent to those skilled in the art from the following detailed description of the preferred embodiment, when read in light of the accompanying drawings.

### BRIEF DESCRIPTION OF THE DRAWINGS

FIG. 1A is a perspective view and cross sectional view of an insect repeller device configured to utilize a vapor emitting device according to the invention.

FIG. 1B is a cross sectional view of a vapor emitting device, according to a first embodiment, including a material reservoir and a wick reservoir.

FIG. 2A-2B are a top perspective view and a cross-sectional view, respectively, of a vapor emitting device, according to a second embodiment, including a material reservoir, a wick reservoir, and a depression.

FIG. 3A-3C illustrate different embodiments of structural configurations for the wick reservoir, according to a third embodiment of a vapor emitting device.

FIG. 4A is a cross-sectional view of a vapor emitting device, according to a fourth embodiment.

FIG. 4B is a cross-sectional view of a vapor emitting device, similar to FIG. 4A.

FIG. 5A-5C are a cross section, perspective view; a cross section, side view, and a plan view of fifth embodiment of a vapor emitting device in accordance with the invention.

### DETAILED DESCRIPTION OF THE PREFERRED EMBODIMENT

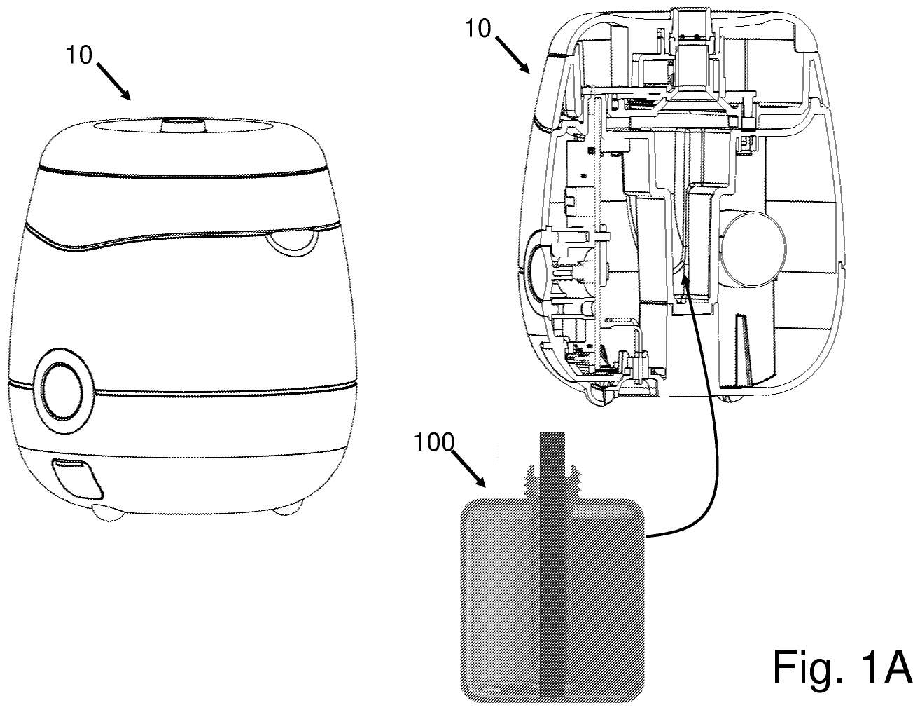

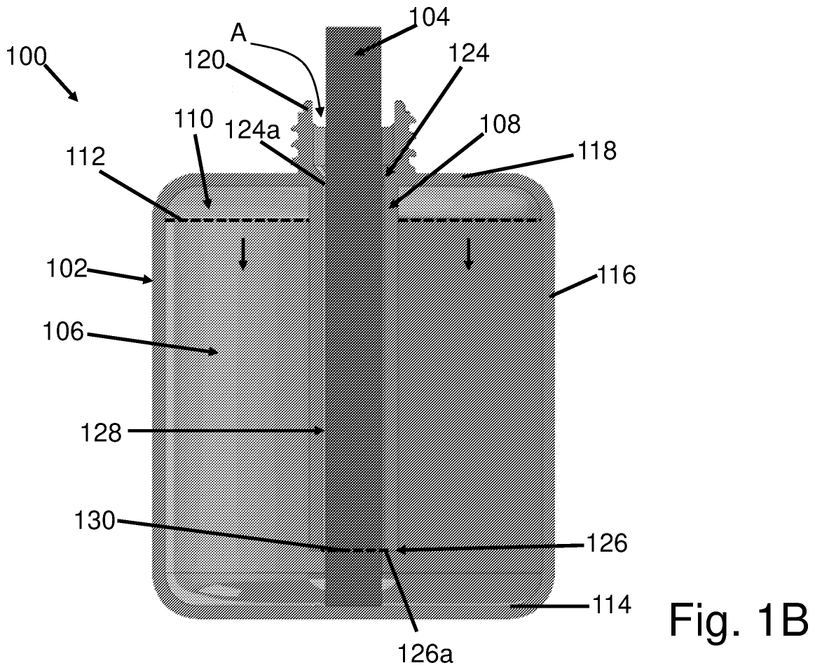

Referring now to the drawings, there is illustrated in FIGS. 1A and 1B an insect repeller device, shown generally at 10, that is configured to accept a vapor emitting device, shown generally at 100. The vapor emitting device 100, according to a first embodiment, is configured as a fluid bottle or refill bottle for the insect repeller device 10. Such a repeller device 10 may be similar to and operate in accordance with the insect repeller devices of PCT/US2021/070978, the disclosure of which is incorporated herein by reference in its entirety. In one aspect of the invention, the vapor emitting device 100 is configured to be used in combination with a heat source of the insect repeller device 10 to generate vapor from a volatilizable material. The vapor emitting device 100 has a main body 102 with a wick 104. The main body 102 can be manufactured from a variety of different materials such as, for example, plastic. Other materials are also contemplated and within the scope of this disclosure. The main body 102 includes a material reservoir 106 and a wick reservoir 108. The material reservoir 106 is configured to contain the volatilizable material and define a vacuum volume 110 or a sufficiently low-pressure zone. In one aspect of the present disclosure, the volatilizable material is a compound that is configured to repel insects. One non-limiting example of the volatilizable material includes metofluthrin. However, it should be appreciated that a skilled artisan can employ alternative compounds that are effective in repelling insects. The vacuum volume 110 is defined by portions of the main body 102 and a first pressure plane 112, illustrated as an upper fluid level within the material reservoir 106. The vacuum volume 110 is configured to support a pressure lower than atmospheric air pressure. Desirably, as will be discussed in further detail, this allows the vapor emitting device 100 to control how much of the volatilizable material comes into contact with the wick 104.

The material reservoir 106 has a base surface 114 surrounded by material reservoir walls 116 and a top surface 118. In one aspect, the top surface 118 can be formed substantially opposite and parallel to the base surface 114. The top surface 118 includes an inlet 120 formed on the top surface 118. The inlet 120 provides an aperture to receive the wick 104 and allow the wick 104 to be disposed within the main body 102. The inlet 120 further permits fluid communication between the vacuum volume 110 and the external atmosphere, as depicted by arrow A in FIGS. 1B, 2B, 3A, and 4A-B.

The wick reservoir 108 receives the wick 104. The wick reservoir 108 extends through the material reservoir 106 and towards the base surface 114 from the inlet 120. In the illustrated embodiment, the wick reservoir 108 is integrally formed with the main body 102. However, it should be appreciated that in some embodiments, such as those shown in FIG. 6, the wick reservoir 108 can be separate from the material reservoir 106. The wick reservoir 108 has a first end 124 and a second end 126. The first end 124 is adjacent to the inlet 120 and includes an inlet orifice 124a. In the illustrated embodiment, the inlet orifice 124a is defined by an outer diameter of the wick 104 and an inner diameter of the wick reservoir 108. The inlet orifice 124a may be configured to contact the wick 104 at discrete points around the wick's diameter and define passages that are in fluid communication with the wick reservoir 108. The second end 126 is adjacent to the base surface 114 and includes a material orifice 126a. It should be appreciated that a diameter of the wick reservoir 108 can be scaled to ensure air can pass between the wick 104 and the wick reservoir 108. The inlet 120 permits fluid communication through the wick reservoir 108 to the outside atmosphere. As will be described below, the material orifice 126a assists in controlling the immersion level of the wick 104 in the volatilizable material for a longer period of time, when in operation.

The wick 104 is disposed through the inlet 120 and the wick reservoir 108 with an air gap 128 formed between the wick 104 and the wick reservoir 108. The air gap 128 functions as a pathway for atmospheric air pressure to contact and exert forces upon the volatilizable material. The atmospheric air pressure and the vacuum level in the vacuum volume 110 establish an equilibrium, which effectively holds the surface of the volatilizable material to a second pressure plane 130 at or near the material orifice 126a. Advantageously, this allows the portion of the wick 104 below the second pressure plane 130 to be submerged in a sufficient and consistent amount of the volatilizable material during operation. With reference to FIG. 1, in certain exemplary conditions, the wick 104 is disposed so that it maintains a clearance with at least a portion of an inner surface of the wick reservoir 108, thereby forming the air gap 128 between the wick 104 and the wick reservoir 108. The air gap 128 may be spaced around the outer surface of the wick 108 or at least a portion of the outer surface of the wick such that fluid communication between the outside atmosphere and the material reservoir can be established. Alternatively, the wick 104 may move within or be shaped to contact the inner surface of the wick reservoir.

As the volatilizable material is drawn up (or wicked) by the wick 104, via capillary action during operation, a pressure imbalance forms between the vacuum volume and the outside atmospheric pressure. This allows air to enter the material reservoir 106 through the inlet orifice 124a along the air gap 128 and out of the material orifice 126a. The air flows up to the vacuum volume 110, which equalizes the pressure differential between the vacuum volume and the external air pressure. This movement of air and equalization of pressure differentials between the vacuum volume 110 and atmospheric conditions permits the volatilizable material to flow by capillary action from the material reservoir 106 through the wick 104. Desirably, this maintains a generally constant level of the volatilizable material around the wick 104 to ensure the portion of the wick 104 below the second pressure plane 130 is submerged with material during operation.

In one aspect of the invention, the distance between the second pressure plane 130 and the base surface 114 may be in a range of about 15% to about 25% of the length of the wick 104. In another aspect, the distance may be in a range of about 20% to about 30% of the length of the wick 104. In yet another aspect, the distance between the second pressure plane 130 and the base surface 114 may be in a range of about 20% to about 40% of the length of the wick 104. In one aspect, the distance between the second pressure plane 130 and the base surface 114 may be about 20% of the wick length 104.

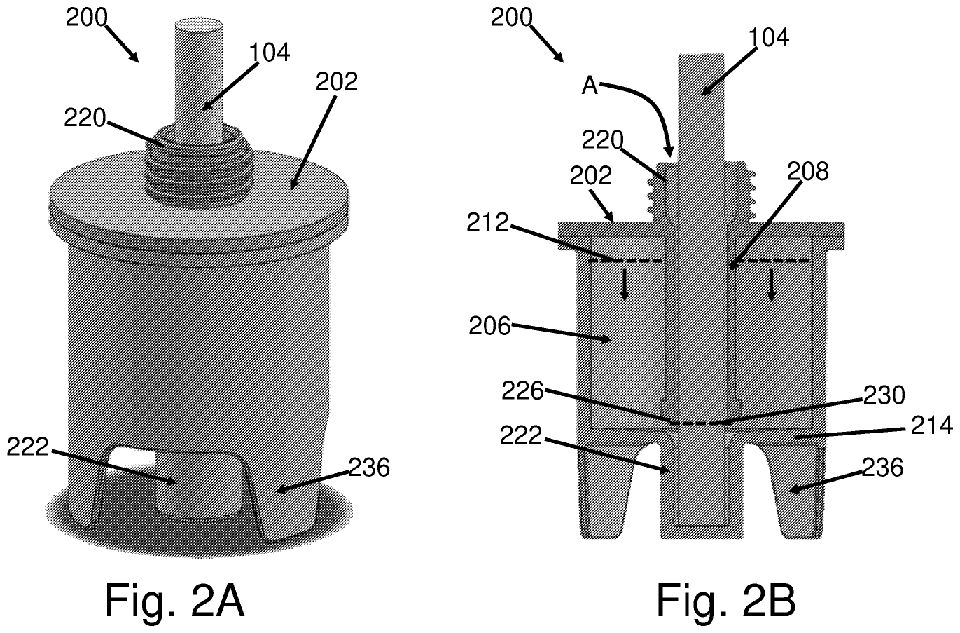

FIG. 2A-2B illustrate a second embodiment of a vapor emitting device 200, illustrated as a fluid bottle. The vapor emitting device 200 includes a main body 202 defining a fluid material reservoir volume contained within a material reservoir 206. The material reservoir 206 includes a depression or cavity that defines a wick immersion volume 222, as shown in FIG. 2B. The wick immersion volume 222 extends from a base surface 214 and can be proximate to or adjacent to a second end 226 of a wick reservoir 208. First and second pressure planes 212 and 230, respectively, are similar to first and second pressure planes 112 and 130, described above. An inlet 220 provides fluid communication with the outside atmosphere to create a pressure equalization with the vacuum volume above the first pressure plane 212, similar to vacuum volume 110. The wick immersion volume 222 is configured to receive the wick 104 and define the volume of volatilizable material in contact with the wick 104 for capillary uptake. In one example, the wick immersion volume 222 maybe large enough to receive at least 20% of a length of the wick 104. Alternatively, the wick immersion volume 222 may accept the wick in a range of about 10% to 20%. In another aspect, the range may be about 18% to 22% or a range of about 20% to about 50%. The length range of the wick immersion volume can be matched to the immersion length of the wick when the wicking action of a particular wick (based on porosity, material composition, fluid viscosity, and fluid attraction to the wick material) is optimized with an immersion of 20% of the wick 104 submerged in the volatilizable material, thereby allowing a more consistent vapor emission rate as the volume of volatilizable material decreases. However, it should be appreciated that one skilled in the art can scale the size of the wick immersion volume 222, as well as the size of the material reservoir 106, to accommodate different wicks and to optimize the wicking action. As shown in FIG. 2A-B, the main body 202 can include additional features, such as optional legs 236, which can assist in keeping the vapor emitting device 200 stable during operation.

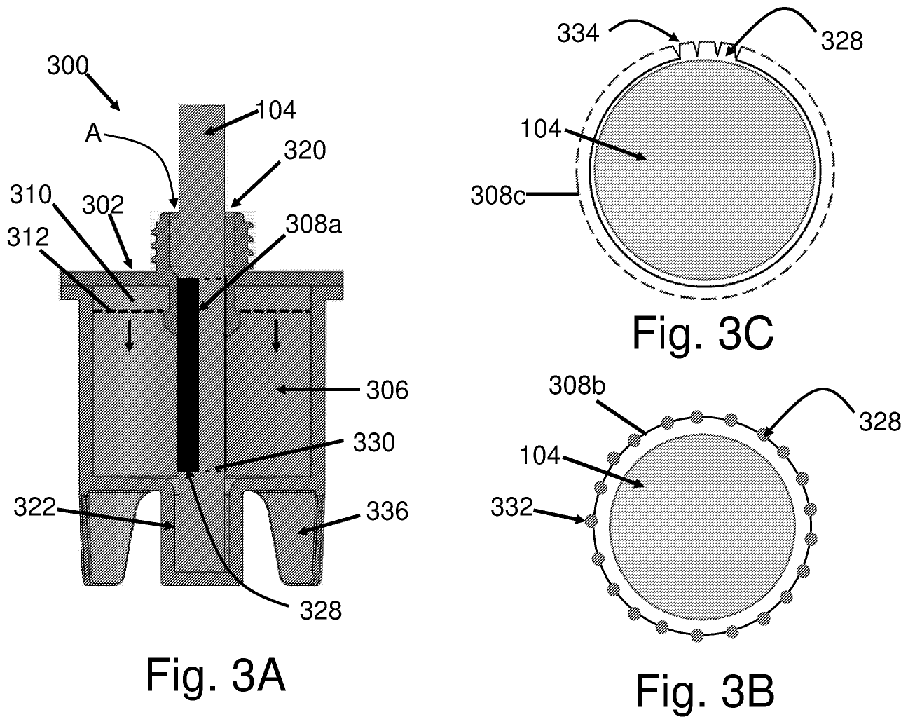

FIG. 3A-3C illustrate a third embodiment of a vapor emitting device 300. The vapor emitting device 300 includes a main body or fluid bottle 302 defining a material reservoir 306. In one aspect, a wick reservoir 308a is a separate sleeve structure disposed around the wick 104 and defines an air gap 328 through a material reservoir 306. The wick 104 can abut the wick reservoir sleeve 308a in discrete locations around the perimeter. The air gap 328 can also be formed using other structures. For example, the air gap 328 can be formed through tubes 332 formed on a wick reservoir 308b adjacent to the wick 104 as shown in FIG. 3B. In another example, the air gap 328 can be formed through flutes 334 formed on a wick reservoir 308c adjacent to the wick to allow air to reach the surface of the volatilizable material defining the first pressure plane 312. As described previously, air is permitted to enter the inlet 320 (as shown by arrow “A”) and travel between the wick reservoir sleeve 308a and the wick 104, as shown in FIG. 3A. Alternative air conduit structures forming the air gaps 328 are shown in FIG. 3B-3C. The wick reservoir sleeves 308a, 308b, and 308c are configured as a substantially impervious sheath or tube formed around the wick 104. This allows air to flow to the surface of the volatilizable material, the first pressure plane 312 defining part of the vacuum volume 310, from the second pressure plane 330, while militating against the volatilizable material from entering through the walls of the wick reservoir 308. The vapor emitting device 300 may include optional legs 336, similar to the legs 236 described above.

In one aspect of the invention, the illustrated embodiment of the vapor emitting device 300 includes a depression or wick uptake cavity forming a wick immersion volume 322. The wick uptake cavity 322 defines a wick immersion depth representing the length of the wick 104 that is in contact with the volatilizable material. The distance between the second pressure plane 330 and the bottom of the wick uptake cavity 322 may be in a range of about 15% to about 25% of the length of the wick 104. In another aspect, the distance may be in a range of about 20% to about 50% of the length of the wick 104. In one aspect, the distance between the second pressure plane 130 and the base surface 114 may be about 20%.

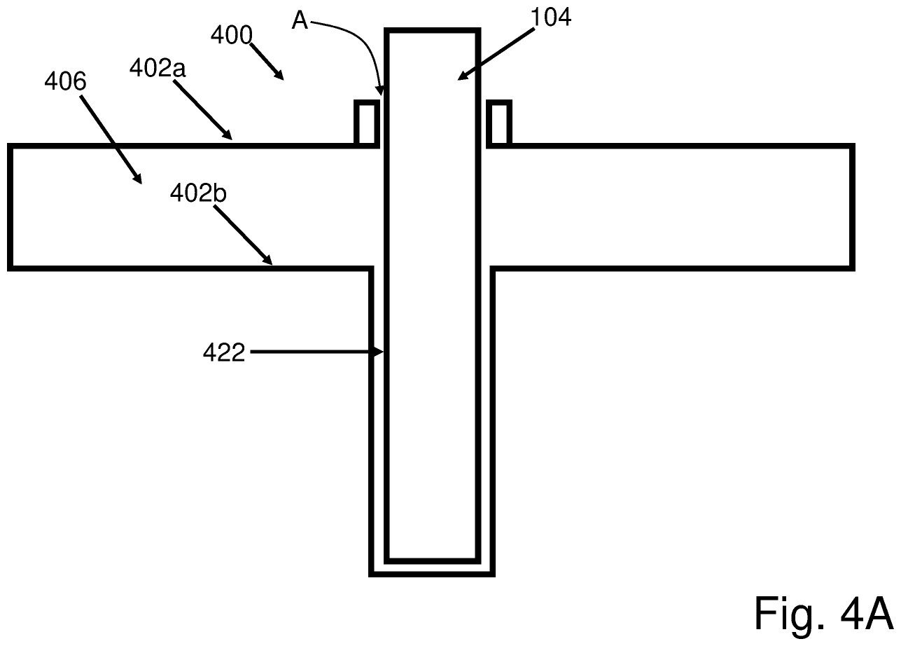

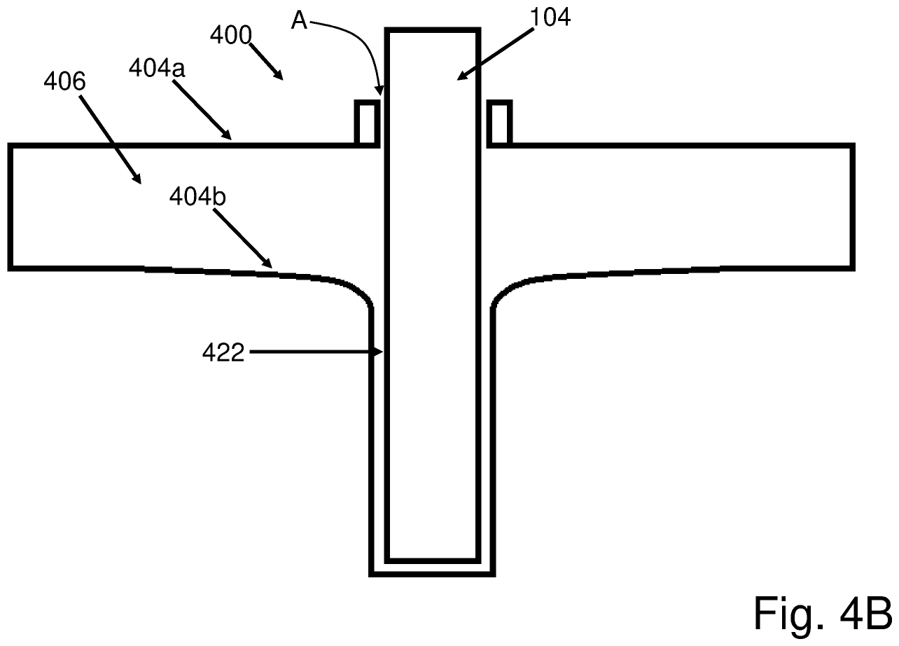

FIGS. 4A and 4B illustrate a fourth embodiment of a vapor emitting device 400. The vapor emitting device 400 includes a main body 402a, shown in FIG. 4A having a generally flat base surface 402b defining a material reservoir 406 encompassing both a volatilizable material volume and a vacuum volume as described above. In a variation of this embodiment, a main body 404a may have a base surface 404b that is sloped, tapered, and/or shaped like a saucer to facilitate fluid flow towards the wick immersion volume 422. In the illustrated embodiment, the main body is larger in diameter or width and shorter in height than the embodiments described above. In one example, the main body may define a diameter (if circular or elliptical) or width to height aspect ratio of about 2:1 to about 5:1. The size of the wick immersion volume 422 is scaled differently according to the size of the material reservoir 406. The wick immersion volume 422 may be in a range of about 50% to about 70% of the wick length such that the majority of the wick is exposed to the wick immersion volume. This further ensures the wick 404 is immersed in the volatilizable material for a longer period of time, when in operation.

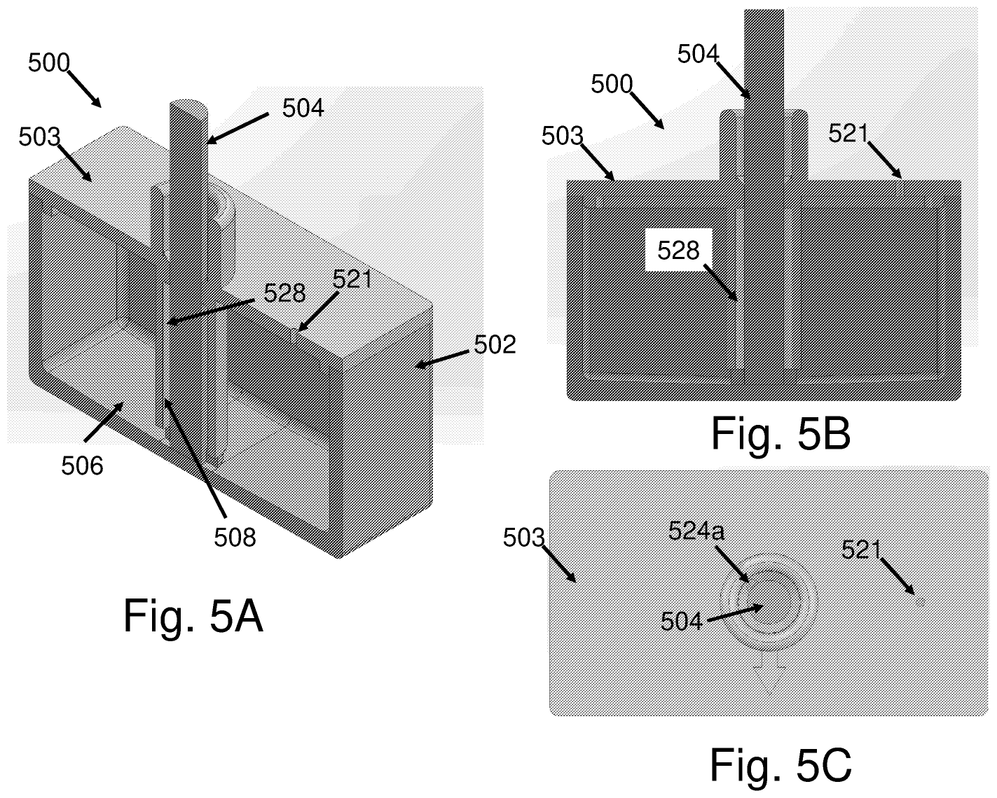

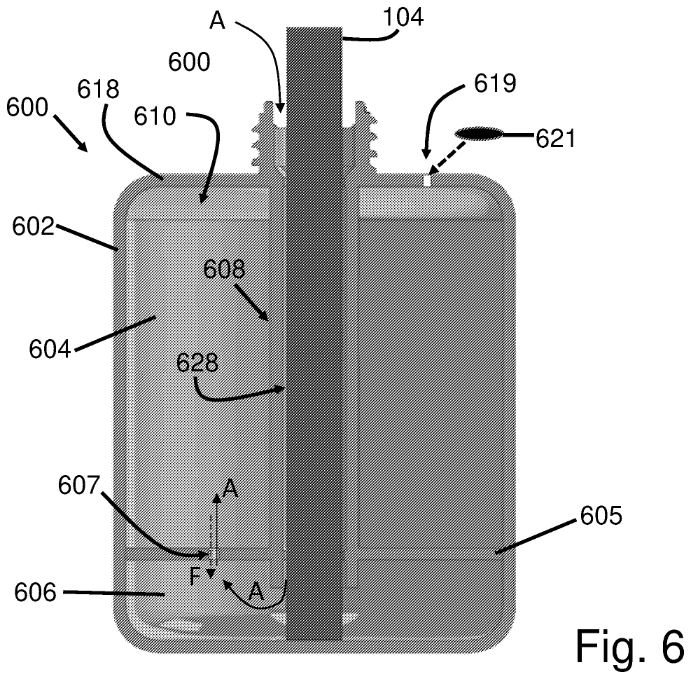

Referring now to FIGS. 5A-5C, there is illustrated another, fifth embodiment of a vapor emitting device shown generally at 500. The vapor emitting device 500 includes a main body 502, a material reservoir 506, and a wick reservoir 508. Operationally, the main body 502 operates in a manner similar to the first embodiment described above. A cover 503 is fixed to the main body 502 to define the material reservoir 506. The cover includes a filling orifice 521 which permits the volatilizable material to be disposed in the material reservoir. The filling orifice 521 is plugged or sealed from the outside atmosphere to prevent air intrusion through the filling orifice once the desired amount of material is provided. An inlet port 524a connects an air gap 528 with the outside atmosphere to permit uptake of volatilizable material by the wick 504.

Referring now to FIG. 6, there is illustrated a sixth embodiment of a vapor emitting device 600 having a two-chamber material reservoir. The vapor emitting device 600 includes a main body 602 defining a first or upper material reservoir 604 and a second or lower material reservoir 606. The upper and lower material reservoirs 604 and 606 are separated by a dividing wall 605 that seals against a wick immersion reservoir 608. The dividing wall 605 includes at least one fluid port 607 to permit fluid to pass from the first material reservoir to the second material reservoir. The wick immersion reservoir 608 extends from an input 620 of the main body 602 and defines an air gap 628 with the wick 104. The air gap 628 is in fluid communication with the outside atmosphere to permit air flow, shown as arrows “A,” to enter the lower material reservoir 606. A vacuum volume 610 is formed between an upper fluid level and an upper wall 618 of the main body as described in conjunction with the first embodiment. An optional fill port 619 may be formed through the upper wall 618 and sealed or plugged with a stopper 621 after the desired amount of fluid is dispensed into the material reservoirs.

As fluid is drawn into the wick 104, the vacuum volume 610 establishes an increasingly larger negative pressure or vacuum acting against the fluid. As the vacuum level increases, air flows through the air gap 628 and bubbles up through the at least one fluid port 607 and into the vacuum volume. This movement of air causes the vacuum level to decrease permitting fluid to flow from the upper material reservoir 604 into the lower material reservoir 606, as shown by arrow “F.” The lower material reservoir 604 defines the wick exposure volume associated with material uptake into the wick. This volume remains generally constant as the material in the upper material reservoir 604 is depleted. Once the upper material volume of fluid is depleted, the fluid in the second material reservoir draws into the wick at a decreasing rate. In one aspect of this embodiment, the second material reservoir may be sized to be 20% of the wick length. Alternatively, the second material reservoir may be sized as a function of the first material reservoir, such as for example 10%, 15%, or 20% of the first material reservoir volume.

The vapor emitting devices described herein may be configured to be received by a heating source as shown in FIG. 1A and described in PCT/US2021/070978 or other heated vaporizing devices known in the art to allow the volatilizable material to be heated to generate vapor. The vapor emitting devices described above improve vapor delivery by defining an immersion volume for the wick 104 that is a generally constant volume of volatilizable material in contact for a longer period of time as the variable volume of fluid in the material reservoir changes during operation. In addition, the wick immersion volumes can also facilitate immersing the wick 104 in a generally constant volume of volatilizable material for a longer period of time.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A vapor emitting device comprising:
a main body having a material reservoir configured to contain a volatilizable material and a vacuum volume, the vacuum volume supporting at least a partial vacuum;
a wick reservoir extending through the material reservoir, the wick reservoir having an inlet in fluid communication with an ambient atmosphere and a material orifice in fluid communication with the volatilizable material; and
a wick disposed through the wick reservoir and into contact with the volatilizable material, the wick and wick reservoir defining an air gap configured to permit the ambient atmosphere to accumulate in the vacuum volume such that the volatilizable material is drawn into the wick.

2. The vapor emitting device of claim 1 wherein the wick reservoir forms a fluid barrier between a portion of the wick and the material reservoir.

3. The vapor emitting device of claim 1 wherein the wick reservoir defines a wick immersion level that controls a length of the wick in contact with the volatilizable material for capillary uptake.

4. The vapor emitting device of claim 3 wherein the wick immersion level is established between a material orifice of the wick reservoir and a submerged end of the wick.

5. The vapor emitting device of claim 4 wherein a first pressure plane is defined between an interface of the volatilizable material and the vacuum volume and a second pressure plane is defined in proximity to the material orifice of the wick reservoir.

6. The vapor emitting device of claim 3 wherein the length of the wick in contact with the volatilizable material is in a range of about 15% to about 25% of an overall length of the wick.

7. The vapor emitting device of claim 4 wherein the material reservoir has a base surface includes a wick immersion volume defining the wick immersion level.

8. The vapor emitting device of claim 7 wherein the wick immersion volume is configured as a depression extending from the base surface.

9. The vapor emitting device of claim 7 wherein the material reservoir is a first material reservoir and the wick immersion volume is a second material reservoir and a dividing wall separates the first material reservoir from the second material reservoir.

10. The vapor emitting device of claim 9 wherein the dividing wall includes at least one fluid port to permit fluid to pass from the first material reservoir to the second material reservoir.

11. The vapor emitting device of claim 1 wherein the wick reservoir is a separate tubular structure extending from the inlet.

12. The vapor emitting device of claim 1 wherein the air gap is defined by one of a plurality of flutes or a plurality of tubes formed on the wick reservoir.

13. The vapor emitting device of claim 1 wherein an insect repeller orients the wick within a heating element to thermally vaporize the volatilizable material in the wick.

14. The vapor emitting device of claim 13 wherein the volatilizable material is metofluthrin.

15. A vapor emitting device, comprising:
a main body having a material reservoir and a wick reservoir, the material reservoir configured to contain a volatilizable material and a vacuum volume, the vacuum volume supporting at least a partial vacuum, the material reservoir having a base surface surrounded by material reservoir walls and a top surface, the base surface including a depression formed on the base surface, the depression defining a wick immersion volume, the top surface including an inlet formed on the top surface, the wick reservoir extending through the material reservoir and towards the base surface from the inlet, the wick reservoir having a first end and a second end, the first end adjacent to the inlet and having an inlet orifice, and the second end adjacent to the wick immersion volume and having a material orifice; and
a wick disposed through the inlet, the wick reservoir, and the wick immersion volume with an air gap formed between the wick and the wick reservoir.

16. The vapor emitting device of claim 15 wherein a plurality of legs extend from the base surface to support the main body.

17. The vapor emitting device of claim 15 wherein the wick reservoir is integrally formed with the top surface.

18. The vapor emitting device of claim 15 wherein the wick reservoir is a separate tubular structure that extends from the inlet.

19. A vapor emitting device, comprising:
a main body having a material reservoir, the material reservoir configured to contain a volatilizable material, the material reservoir having a base surface surrounded by material reservoir walls with a top surface, the base surface including a depression formed on the base surface, the depression defining a wick immersion volume, the top surface including an inlet formed on the top surface; and
a wick disposed through the inlet and the wick immersion volume.

20. The device of claim 19, wherein the base is sloped towards the wick immersion volume.
